(19) United States
(12) Patent Application Publication
(10) Pub. No.: US 2008/0194628 A1
(43) Pub. Date: Aug. 14, 2008

(54) Donepezil Salts Suitable for the Preparation of Pharmaceutical Compositions

(76) Inventors: Tibor Mezei, Budapest (HU); Gyula Simig, Budapest (HU); Gyula Lukacs, Budapest (HU); Marta Porcs-Makkay, Pomaz (HU); Balazs Volk, Budapest (HU); Eniko Molnar, Erd (HU); Valeria Hofmanne Fekete, Budapest (HU)

Correspondence Address: K.F. ROSS P.C., 5683 RIVERDALE AVENUE, SUITE 203 BOX 900, BRONX, NY, 10471-0900, US

(21) Appl. No.: 11/662,867

(22) PCT Filed: Dec. 9, 2005
(86) PCT No.: PCT/HU2005/000102

§ 371 Date: Oct. 3, 2007

(30) Foreign Application Priority Data
Sep. 15, 2004 (HU) P0401850

(51) Int. Cl. A61K 31/445; C07D 211/06; A61P 25/00

(52) U.S. Cl. 514/319; 546/206

(57) ABSTRACT

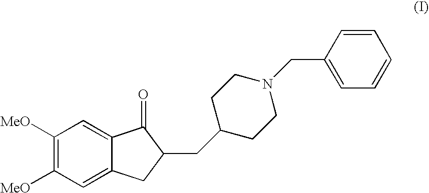

The invention relates to donepezil salts formed with organic acids and a process for the preparation thereof. Said salts can be used for the preparation of pharmaceutical compositions. The invention also relates to a process for the preparation of said salts, pharmaceutical compositions containing them and the use of said compounds for the treatment of diseases.

### FIELD OF THE INVENTION

The invention relates to donepezil salts useful for the preparation of pharmaceutical compositions. Furthermore, the invention also relates to a process for the preparation of said salts, pharmaceutical compositions containing them and the use of said compounds for the treatment of diseases.

More particularly the present invention is concerned with salts of 1-benzyl-4-[(5,6-dimethoxy-1-indanon-2-yl)-methyl]-piperidine) (INN name: donepezil) of the formula (I),

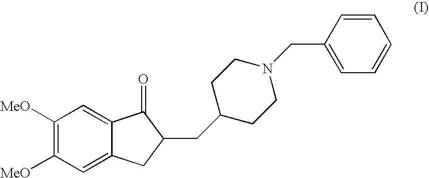

formed with organic acids of the general formula H—X, wherein X stands for an organic acid radical.

### TECHNICAL BACKGROUND OF THE INVENTION

Donepezil is a pharmaceutical ingredient for the treatment of senile dementia, which is used in the form of the hydrochloride salt for the preparation of pharmaceuticals.

The quick increase in the ratio of the aged within the population requires the development of efficient therapies for the treatment and prophylaxis of senile dementia developing e.g. as a consequence of Alzheimer's disease.

Several ingredients have been on trial for the treatment of dementia, but only a partial result could be achieved by using them. However, it has been observed that in the organism of patients with Alzheimer's disease the acetylcholine concentration is considerably lower than in healthy persons. On this basis it could be concluded that a method for the treatment of said disease could be the administration of pharmaceuticals increasing the level of acetylcholine, especially in the brain. Practically two ways are provided for this purpose.

According to one of the two solutions acetylcholine precursors are delivered into the organism, from which substances acetylcholine is formed by complicated biochemical processes. Thus these substances can be considered as pre-drugs. By administering them a higher acetylcholine concentration can be achieved in the organism.

According to the other solution a substance inhibiting the enzyme responsible for the decomposition of acetylcholine, that is a so-called acetylcholine esterase inhibitor is delivered into the organism. In this way the decomposition of acetylcholine is inhibited. Such acetylcholine esterase inhibitors are physostigmine and tetrahydroacridine. These ingredients possess, however, unpleasant side-effects, as they inhibit the decomposition of acetylcholine not only in the brain but in the whole organism.

Donepezil is the first long-lasting, strong and highly selective acetylcholine esterase inhibiting pharmaceutical ingredient which enhances the level of acetylcholine in the brain in a much larger extent than in other parts of the organism. The efficacy of this substance in case of memory losses and its clinical applicability more advantageous than that of physostigmine has been demonstrated by model experiments.

Donepezil is suitable for the treatment and prophylaxis of cerebral diseases that can be attributed to a deficiency of acetylcholine. Such diseases include e.g. Alzheimer's disease, Huntington syndrome, ataxia or Pick's disease.

Donepezil is provided in Hungarian patent specification No. 214,592. The medical use of its salt formed with hydrogen chloride is disclosed in Hungarian patent specification No. 211,165. Four further polymorphic crystalline forms of the hydrochloride salt of this active ingredient have been invented and applied for patent protection. Said crystalline forms different from that specified in the basic patent are provided in the International patent application No. WO97/46526.

The pharmaceuticals put on the market have to meet a number of requirements raised by different authorities. Said requirements are more and more strict and are to be evidenced by appropriate documentation. A part of the specifications relates to the active ingredient, the other part is related to the pharmaceutical composition, which are closely interlinked during the development of the composition and the evaluation of the marketing documentation.

The strictest requirements towards pharmaceutically active ingredients are those wherein purity is involved. In most cases the active ingredients are organic bases of high molar weight, which are insoluble in water and unwettable with water. The hydrophobic property of the active ingredient is problematical, especially when formulating the dosage units. It is expedient to convert the basic active ingredient to a salt with a pharmaceutically acceptable organic salt and to use the thus-obtained salt for the preparation of the pharmaceutical composition. A further advantage of the application of salts reside in the fact that they are more freely soluble in water and much more readily wettable with water than the corresponding bases. Besides, due to their melting point higher than that of the bases they can be purified easily and effectively.

The most important requirement raised towards pharmaceutical compositions getting on the market is that they should remain stable when examined according to pharmacopoeal specifications. Stability means that the decrease of the active ingredient in the pharmaceutical composition during manufacturing or storage will not exceed the permissible level.

To insure the stability of a pharmaceutical composition is a complex task as a consequence of some mechanical effects and heat occurring in the manufacturing process. During the preparation of a pharmaceutical composition it often happens that substances suitable for the formation of a big specific surface and possibly swelling upon the effect of humidity are used. On a big surface certain chemical processes—which may be undesired decomposition, oxidation or hydrolysis reactions—may be much quicker, because in such cases the active ingredient is in contact with the air and humidity on a big surface. This is the case especially when pharmaceutical ingredients of small particle size are applied, that is the active substance is in micronized form.

In order to evidence stability of the pharmaceutical compositions they are subjected to strict examinations according to the requirements of licensing authorities, considering that the decomposition reactions can practically never be predicted. The essential part of the stability examinations constitutes storing the pharmaceutical composition at a constant high temperature (between 50° C. and 70° C.) under a high humidity content, determining the active ingredient content at pre-determined times (usually after several months) and carrying out quantitative and qualitative analysis for the impurities formed in the composition as a result of decomposition processes. For this purpose the structure of the most important impurities being present expectedly in an amount exceeding a certain level is to be determined, and a sample applicable as a reference substance is to be synthesised from them.

During the stability examinations of tablets containing donepezil hydrochloride several impurities being present in different concentrations appear, which can be detected by mass spectrometry (MS), identified and their concentration can be determined by high-efficiency liquid chromatography (HPLC).

In order to evidence that the impurity with identified structure and the synthesised comparative substance are identical, separate experiments, such as MS or connected HPLC-MS methods have to be carried out.

In the course of stability examinations carried out with tablets having different composition but containing as active ingredient donepezil hydrochloride different impurities can be detected in the samples. The inventors have determined the molar weight of said impurities by mass spectrometry (MS). One the basis of the MS examination one of the impurities was supposed to be the compound of the formula (III)

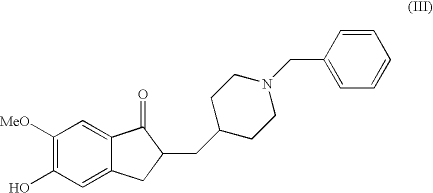

obtained from donepezil by partial demethylation. The inventors have prepared (±)-2-[(1-benzyl-4-piperidil)methyl]-5-hydroxy-6-methoxy-1-indane of the formula (III) and evidenced by HPLC method that the same compound is formed in the course of the stability examinations of tablets containing donepezil hydrochloride.

When studying the technical literature it has been found that aromatic methoxy groups at the ortho position, which are present also in the donepezil molecule, are liable to partial hydrolysis in the present of a strong mineral acid. Demethylation of aromatic methoxy derivatives is carried out with aqueous hydrogen chloride (Pyman, J. J. Chem. Soc. 97, 275 (1910)) or with hydrogen bromide in acetic acid (Tomit et al., Yakugaku Zasshi, 76, 1122 (1956)), at an elevated temperature. Under vigorous conditions usually both methoxy groups are split off, but surprisingly one of the methoxy groups at the ortho position is demethylated into a hydroxy group even under very mild conditions, depending on the substituents of the aromatic ring. According to the literature partial demethylation of aromatic methoxy groups at the ortho position in the presence of mineral acids may occur even at room temperature (Blaskó, G. et al., Tetrahedron Lett. 22, 3135-3138 (1981)). The o-demethylation process experienced when reacting donepezil with mineral acids takes place also under specific conditions characteristic of tablets. For the purpose of comparison the salts of donepezil formed with hydrogen bromide and sulfuric acid have also been prepared according to comparative Examples 1 and 2 (see below). It could be established that the stability of tablets prepared from these salts is practically identical to that of the tablet prepared from the hydrochloride salt.

The aim of the invention was to prepare donepezil salts suitable for the preparation of stable pharmaceutical compositions and substantially devoid of (±)-2-[(1-benzyl-4-piperidil)methyl]-5-hydroxy-6-methoxy-1-indanone of the formula (III).

### SUMMARY OF THE INVENTION

The invention is based on the surprising recognition that in case of using a salt of donepezil formed with an organic acid for the preparation of tablets, the compound of the formula (III) cannot be detected in the course of the stability examinations.

### DETAILS OF THE INVENTION

According to an aspect of the present invention there are provided donepezil salts of the general formula (II),

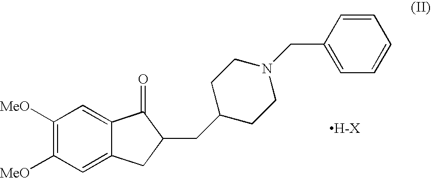

wherein X stands for the radical of an organic acid, such as formic, acetic, propionic, maleic, fumaric, succinic, lactic, malic, tartaric, citric, ascorbic, malonic, oxalic, mandelic, glycolic, phthalic, benzenesulfonic, toluenesulfonic, naphthalenesulfonic or methanesulfonic acid, preferably fumaric, maleic, methanesulfonic, benzenesulfonic or toluenesulfonic acid.

which exert more advantageous stability characteristics than the donepezil salts formed with inorganic acids known from the literature.

Among the donepezil salts according to the invention formed with organic acids the fumarate salt has outstanding characteristics. The physical properties, stability and solubility of this salt are particularly advantageous for the preparation of pharmaceutical compositions. Its solubility in water is almost identical to that of the hydrochloride salt known from the literature. Its melting point is above 150° C., which is particularly advantageous for the preparations of medicines, such as tablets. The fumarate salt of donepezil according to the invention is substantially devoid of the impurity of the formula (III).

According to another aspect of the present invention there is provided a process for the preparation of donepezil salts of the general formula (II) formed with organic acids, which comprises reacting donepezil base in an appropriate organic solvent with the desired organic acid, isolating the crystallizing donepezil salt and optionally washing it with an organic solvent.

As solvent a C₁₋₄ alcohol, ether or ester, preferably diethyl ester, ethyl acetate, methanol, ethanol, 2-propanol or mixtures thereof may be used.

The organic acid serving for salt formation is used in an amount of 1.0-1.3 molar equivalent, preferably in equimolar amount, related to the amount of the donepezil base.

According to a further aspect of the present invention there are provided pharmaceutical compositions containing as active ingredient a compound of the general formula (II) in admixture with one or more carrier(s) or auxiliary substance(s) conventionally applied in the pharmaceutical industry. The pharmaceutical compositions according to the invention are practically devoid of (±)-2-[(1-benzyl-4-piperidil)methyl]-5-hydroxy-6-methoxy-1-indanone of the formula (III).

According to a still further aspect of the present invention there is provided a process for the preparation of the pharmaceutical compositions containing as active ingredient a donepezil salt of the general formula (II), which comprises admixing said active ingredient with one or more carrier(s) or auxiliary substance(s) conventionally applied in the pharmaceutical industry and bringing the mixture to galenic form.

The pharmaceutical compositions according to the invention usually contain 0.1-95% by weight, preferably 1-50% by weight, particularly 5-30% by weight of active ingredient.

The pharmaceutical compositions of the present invention may be suitable for oral (e.g. powders, tablets, coated tablets, capsules, microcapsules, pills, solutions, suspensions or emulsions), parenteral (e.g. injection solutions for intravenous, intramuscular, subcutaneous or intraperitoneal use), rectal (e.g. suppositories) transdermal (e.g. plasters) or local (e.g. ointments or plasters) administration or for the application in form of implants. The solid, soft or liquid pharmaceutical compositions according to the invention may be produced by methods conventionally applied in the pharmaceutical industry.

The solid pharmaceutical compositions for oral administration containing the compounds of the general formula (I) or pharmaceutically acceptable acid addition salts thereof may comprise fillers or carriers (such as lactose, glucose, starch, calcium phosphate, microcrystalline cellulose), binding agents (such as gelatine, sorbite, polyvinyl pyrrolidone), disintegrants (such as croscarmelose, Na-carboxymethyl cellulose, crospovidone), tabletting auxiliary agents (such as magnesium stearate, talc, polyethylene glycol, silicic acid, silicon dioxide) and surface-active agents (e.g. sodium lauryl sulfate).

The liquid compositions suitable for oral administration containing the compounds of the general formula (II) can be solutions, suspensions or emulsions. Such compositions may contain suspending agents (e.g. gelatine, carboxymethyl cellulose), emulsifiers (e.g. sorbitane mono-oleate, solvents (e.g. water, oils, glycerol, propyleneglycol, ethanol), buffering agents (e.g. acetate, phosphate, citrate buffers) and preservatives (e.g. methyl-4-hydroxybenzoate etc.).

Liquid pharmaceutical compositions suitable for parenteral administration are generally sterile isotonic solutions optionally containing, in addition to the solvent, buffering agents and preservatives.

Soft pharmaceutical compositions containing as active ingredient a compound of the general formula (I) or a pharmaceutically acceptable acid addition salt thereof, such as suppositories, contain the active ingredient evenly dispersed in the basic material of the suppository (e.g. in polyethylene glycol or cocoa butter).

The pharmaceutical compositions according to the present invention containing a compound of the general formula (II) can be prepared by known methods of the pharmaceutical industry. The active ingredient is admixed with pharmaceutically acceptable solid or liquid carriers and/or auxiliary agents and the mixture is brought to galenic form. The carriers and auxiliary agents together with the methods which can be used in the pharmaceutical industry are disclosed in the literature (Remington's Pharmaceutical Sciences, Edition 18, Mack Publishing Co., Easton, USA, 1990).

The pharmaceutical compositions according to the present invention contain generally a dosage unit of the active ingredient of the general formula (II).

According to a still further aspect of the present invention there is provided the use of the compounds of general formula (II) as pharmaceutical ingredients.

Further details of the present invention are to be found in the following Examples without limiting the scope of protection to the said Examples.

### EXAMPLE 1

Preparation of Donepezil Fumarate

Into an equipment enabling vigorous stirring 550 ml of anhydrous ethanol are measured, and 38.0 g (0.10 mole) of donepezil base are dissolved in it under stirring. To the solution 11.6 g (0.10 mole) of fumaric acid are added at 60° C., the solution is heated to boiling point, clarified with 2.5 g of activated charcoal and allowed to cool to room temperature within 2 hours. Crystallization starts at 60° C. The suspension is stirred at 0° C. for 2 hours, filtered and washed on the filter with 0° C. ethanol until free of the mother liquor.

Yield: 47.2 g (95.4%) of white crystals

Melting point: 170-171° C.

Analysis for the formula C₂₄H₂₉NO₃.C₄H₄O₄ (4955):

Calculated C, 67.86%; H, 6.71%; N, 2.83%.

Found: C, 67.74%; H, 6.65%; N, 2.83%.

According to HPLC the purity of the product amounts to 99.8%.

### EXAMPLE 2

Preparation of Donepezil Maleinate

Into an equipment enabling vigorous stirring 100 ml of 2-propanol are measured, and 7.6 g (20 mmoles) of donepezil base are dissolved in it under stirring. To the solution 2.32 g (20 mmoles) of maleic acid are added at 60° C., the solution is heated to boiling point, clarified with activated charcoal and allowed to cool to room temperature within 1 hour. The suspension is stirred at 0° C. for 2 hours, filtered and washed on the filter with 0° C. ethyl acetate until free of the mother liquor.

Yield: 9.04 g (91.2%) of white crystals.

Melting point: 116-118° C.

Analysis for the formula C₂₄H₂₉NO₃.C₄H₄O₄ (495.5):

Calculated C, 67.86%; H, 6.71%; N, 2.83%.

Found: C, 67.24%; H, 6.85%; N, 2.79 %.

According to HPLC the purity of the product amounts to 99.8%.

### EXAMPLE 3

Preparation of Donepezil Methanesulfonate

Into an equipment enabling vigorous stirring 100 ml of 2-propanol are measured, and 7.6 g (20 mmoles) of donepezil base are dissolved in it under stirring. To the solution 1.92 g (20 mmoles) of methanesulfonic acid is added, the solution is heated to boiling point, clarified with 2.5 g of activated charcoal and allowed to cool to room temperature. The suspension is filtered at 0° C. and washed on the filter with 0 ° C. ethyl acetate until free of the mother liquor.

Yield: 9.34 g of (89.2%) of white crystals

Melting point: 180-182° C.

Analysis for the formula C₂₅H₃₃NO₆S (475.6):

Calculated C, 63.14%; H, 6.99%; N, 2.95%; S, 6.74%.

Found: C, 62.98%; H, 7.02%; N, 2.94%; S, 6.70%.

### EXAMPLE 4

Preparation of Donepezil Benzenesulfonate

Into an equipment enabling vigorous stirring 100 ml of 2-propanol are measured, and 7.6 g (20 mmoles) of donepezil base are dissolved in it under stirring. To the solution 3.16 g (20 mmoles) of benzenesulfonic acid are added, the solution is heated to boiling point, clarified with 2.5 g of activated charcoal and allowed to cool to room temperature. The suspension is filtered at 0° C. and washed oh the filter with 0° C. ethyl acetate until free of the mother liquor.

Yield: 9.41 g of (87.5%) of white crystals

Melting point: 175-176° C.

Analysis for the formula C₃₀H₃₅NO₆S (537.7):

Calculated C, 67.02%; H, 6.56%; N, 2.61%; S, 5.96%.

Found: C, 66.94%; H, 6.53%; N, 2.58%; S, 5.91%.

### EXAMPLE 5

Preparation of Donepezil p-toluenesulfonate

Into an equipment enabling vigorous stirring 100 ml of 2-propanol are measured, and 7.6 g (20 mmoles) of donepezil base are dissolved in it under stirring. To the solution 3.45 g of (20 mmoles) of methanesulfonic acid are added, the solution is heated to boiling point, clarified with activated charcoal and allowed to cool to room temperature. The suspension is filtered at 0° C. and washed on the filter with 0° C. ethyl acetate until free of the mother liquor.

Yield: 9.29 g (84.2%) of white crystals

Melting point: 171-173° C.

Analysis for the formula C₃₁H₃₇NO₆S (551.7):

Calculated C, 67.49%; H, 6.76%; N, 2.54%; S, 5.81%.

Found: C, 67.54%; H, 6.83%; N, 2.54%; S, 5.76%.

### EXAMPLE 6

### Preparation of (±)-2-[(1-benzyl-4-piperidinyl)methyl]-5-hydroxy-6-methoxy-1-indanone hydrochloride [the compound of formula (III)]

7.6 g (20 mmoles) of donepezil base are stirred in a mixture of 50 ml of 48% aqueous hydrogen bromide and 10 ml of acetic acid in a water bath for 20 hours. The solution is poured onto 500 g of ice, neutralized with potassium carbonate, the product is extracted with ethyl acetate and the solution is evaporated at reduced pressure. From the residual oil hydrogen chloride salt is formed in a 5:1 (v/v) mixture of diethyl ether and 2-propanol.

Yield: 2.85 g (35.4%) of white crystals.

Melting point: 159-160° C.

Analysis for the formula C₂₃H₂₈ClNO₃ (401.9):

Calculated: C, 68.73%; H, 7.02%; N, 3.48%; Cl, 8.82%.

Found: C, 68.63%; H, 7.12%; N, 3.45%; Cl, 8.95%.

### EXAMPLE 7

### Preparation of (±)-2-[(1-benzyl-4-piperidinyl)methyl]-5-hydroxy-6-methoxy-1-indanone hydrochloride [a compound of the general formula (III)]

7.6 g (20 mmoles) of donepezil base are stirred in a mixture of 50 ml of 36.5% aqueous hydrogen bromide and 10 ml of acetic acid in a water bath of 80° C. for 24 hours. The solution is poured onto 500 g of ice, neutralized with potassium carbonate, the product is extracted with ethyl acetate and the solution is evaporated at reduced pressure. From the residual oil hydrochloride salt is formed in ethyl acetate.

Yield: 2.10 g of (26.1%) of white crystals

Melting point: 158-160° C.

Analysis for the formula C₂₃H₂8ClNO₃ (401.9):

Calculated: C, 68.73%; H, 7.02%; N, 3.48%; Cl, 8.82%.

Found: C, 68.55%; H, 6.94%; N, 3.54%; Cl, 8.71%.

### EXAMPLE 8

Preparation of a Pharmaceutical Composition

For the preparation of tablets with a total weight of 100 mg containing 5 mg of active ingredient the following substances are measured (related to one tablet):

| | |
|---|---|
| donepezil fumarate | 5 mg |
| lactose | 47 mg |
| corn starch | 47 mg |
| magnesium stearate | 1 mg |

The powder mixture is homogenized and compressed into tablets.

### EXAMPLE 9

Preparation of a Pharmaceutical Composition

For the preparation of tablets with a total weight of 100 mg containing 10 mg of active ingredient the following substances are measured (related to one tablet):

| | |
|---|---|
| donepezil fumarate | 10 mg |
| lactose | 30 mg |
| corn starch | 59 mg |
| magnesium stearate | 1 mg |

The powder mixture is homogenized and compressed into tablets.

### EXAMPLE 10

Preparation of a Pharmaceutical Composition

For the preparation of tablets with a total weight of 100 mg containing 25 mg of active ingredient the following substances are measured (related to one tablet):

| | |
|---|---|
| donepezil fumarate | 25 mg |
| lactose | 50 mg |
| corn starch | 24 mg |
| magnesium stearate | 1 mg |

The powder mixture is homogenized and compressed into tablets.

### EXAMPLE 11

### Comparative Experiment

Preparation of Donepezil Hydrobromide

Into an equipment enabling vigorous stirring 100 ml of 2-propanol are measured, and 7.6 g (20 mmoles) of donepezil base are dissolved in it. To the solution 2-propanol containing 1.62 g (20 mmoles) of hydrogen bromide is added. The suspension is filtered at 0° C. and washed on the filter with ethyl acetate until free of the mother liquor.

Yield: 8.28 g of (89.9%) of white crystals

Melting point: 246-247° C.

Analysis for the formula C₂₄H₃₀BrNO₃ (460.7):

Calculated: C, 62.61%; H, 6.57%; Br, 17.35%; N, 3.04%.

Found: C, 62.33%; H, 6.55%; Br, 17.57%; N, 3.00%.

### EXAMPLE 12

### Comparative Example

Preparation of donepezil sulfate (1:1)

Into an equipment enabling vigorous stirring 100 ml of 2-propanol are measured, and 7.6 g (20 mmoles) of donepezil base are dissolved in it. To the solution 2-propanol containing 2.45 g (2 5 mmoles) of sulfuric acid is added. The suspension is stirred at 0° C. for 2 hours and washed on the filter with ethyl acetate until free of the mother liquor.

Yield: 8.83 g of (92.4%) of white crystals

Melting point: 190-195° C.

Analysis for the formula C₂₄H₃₁NO₇S (477.6):

Calculated: C, 60.36%; H, 6.54%; N, 2.93%; S, 6.71%.

Found: C, 59.95%; H, 6.52%; N, 2.87%; S, 6.64%.

What we claim is:

1. Acid addition salts of the general formula (II) of donepezil [(±)-2-[(1-benzyl-4-piperidineil)methyl]-5, 6-dimethoxy-1-indanonel 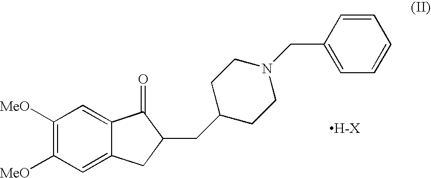
formed with organic acids, wherein X stands for an organic acid radical.

2. Salts of the general formula (II) according to claim 1, wherein X stands for the radical of an organic acid, such as formic, acetic, propionic, maleic, fumaric, succinic, lactic, malic, tartaric, citric, ascorbic, malonic, oxalic, mandelic, glycolic, phthalic, benzenesulfonic, toluenesulfonic, naphthalenesulfonic or methanesulfonic acid, preferably fumaric, maleic, methanesulfonic, benzenesulfonic or toluenesulfonic acid.

3. Donepezil fumarate (1:1).

4. Donepezil maleinate (1:1).

5. Donepezil methanesulfonate.

6. Donepezil benzenesulfonate.

7. Donepezil toluenesulfonate.

8. Acid addition salts of donepezil formed with organic acids as claimed in any of claims 1 to 7, which are substantially devoid of (±)-2-[(1-benzyl-4-piperidinyl)methyl]-5-hydroxy-6-methoxy-1-indanone of the formula (III).

9. A process for the preparation of donepezil salts as claimed in any of claims 1 to 7, which comprises reacting donepezil base in a suitable organic solvent with the desired organic acid, separating the thus-obtained donepezil salt and optionally washing it with an organic solvent.

10. A process as claimed in claim 9, which comprises using said organic acid in an amount of 1.0-1.3 molar equivalent, preferably 1.0 molar equivalent.

11. A process as claimed in claims 9 and 10, which comprises using as solvent a C₁₋₄ alcohol, ether or ester, preferably diethyl ether, ethyl acetate, methanol, ethanol, 2-propanol or mixtures thereof.

12. Pharmaceutical compositions comprising as active ingredient donepezil salts according to claims 1 to 8 together with one or more carrier(s) or auxiliary substance(s) conventionally applied in the pharmaceutical industry

13. A process for the preparation of pharmaceutical compositions according to claim 12, which comprises admixing a compound of the general formula (II) according to any of claims 1 to 8 with a pharmaceutically acceptable carrier and optionally other auxiliary agent and bringing the mixture to galenic form.

14. Use of a donepezil salt according to any of claims 1 to 8 for the preparation of a pharmaceutical composition suitable for the prophylaxis or treatment of diseases in connection with a cerebral deficiency of acetylcholine, Alzheimer's disease or senile dementia.

15. A method for the prophylaxis or treatment of diseases in connection with a cerebral deficiency of acetylcholine, Alzheimer's disease or senile dementia, which comprises administering to the patient at least one donepezile salt of the general formula (II) as claimed in any of claims 1 to 8 in a pharmaceutically effective amount to a patient in need of such a treatment.

16. (±)-2-[(1-benzyl-4-piperidinyl)-methyl]-5-hydroxy-6-methoxy-1-indanone of the formula (III).

17. Salts of (±)-2-[(1-benzyl-4-piperidinyl)methyl]-5-hydroxy-6-methoxy-1-indanone formed with mineral acids, such as sulfuric acid, hydrogen chloride or hydrogen bromide.
